(19) United States
(12) Patent Application Publication
(10) Pub. No.: US 2005/0013787 A1
(43) Pub. Date: Jan. 20, 2005

(54) Method and composition for treating hair

(76) Inventor: Melody M. Benton, Kennard, TX (US)

Correspondence Address: JOHN F. BRYAN, P.O. BOX 1987, PLANO, TX, 75086, US

(21) Appl. No.: 10/619,222

(22) Filed: Jul. 14, 2003

(51) Int. Cl. A61K 7/06; A61K 7/11

(52) U.S. Cl. 424/70.14

(57) ABSTRACT

A hair conditioning shampoo having a commercially available shampoo, with anionic and nonionic surfactants in aqueous solution with a thickening agent in combination with an effective amount of food grade gelatin, having physical properties of between 200 and 250 grams bloom; approximately 32 mps viscosity and preferably, a pH value of between 4.5 and 5.8, so as to provide both detangling and rejuvenation of dry, brittle hair.

### TECHNICAL FIELD

The present invention relates to the field of hair-care and more particularly, to remedial hair conditioning shampoos such as are used on dry, brittle hair for prevention of breakage.

### BACKGROUND

The hair care industry offers many chemically based treatments for enhancing the appearance of hair. Particularly popular among these treatments for appearance conscious women are permanent waves, hair coloring and bleaching. All of these treatments, along with hair dryers and exposure to the elements, conspire to cause dry brittle hair with split ends. This condition is treated, with varying degrees of success, by use of products known as hair conditioners or hair conditioning shampoos. The basic formulation of commercially available hair conditioning products generally comprises a low viscosity solution of about 95% water; about 2% of a detangling agent such as a quaternary ammonium compound or an acylated peptide; about 1% of an emulsifying agent such as a polyhydric alcohol or a polyalkylene glycol ether; about 1% of a germicide as a preservative agent, about ½% of a stabilized fragrance; and about ½% of an antioxidant such as ascorbic acid. A thickening agent is added to the basic conditioner formulation to provide suitable viscosity and “feel”. As an example, this thickener may comprise a miscible propylene glycol based solution.

As a practical matter, the primary function of such conditioners is detangling, although supplementary additives may also be added for improved hair sheen, hair strengthening and moisturizing. Some skilled in the art consider the addition of about 1% collagen, a naturally occurring animal gelatinous material, to be desirable for hair strengthening because, as a natural material, collagen is thought to penetrate and strengthen the hair shaft.

The basic formulation of commercially available shampoo products generally comprises a low viscosity solution of about 75% water; about 20% anionic and nonionic surfactants; about 1% germicidal preservatives; about ½% stabilized fragrance; and about ½% antioxidants such as ascorbic acid; and about 1% of an opacifying agent such as glycol; and a minor amounts of fragrance. As described above for conditioners, a miscible thickening agent is added to the basic shampoo formulation for suitably thicker viscosity and “feel”.

A “hair conditioning shampoo” is essentially a combination of the two above described formulations. While such conditioners and hair conditioning shampoos have been generally acceptable for purposes of detangling and treating dry, brittle hair conditions, hair can become so severely damaged that even multiple treatments are ineffective.

Therefore, it is a first object of the present invention to provide effective treatment for severe cases of dry, brittle hair. A second object is to provide a hair conditioning shampoo for such a treatment in an embodiment that can be prepared by a lay person, without access to laboratory facilities.

### SUMMARY OF THE INVENTION

The present inventions contemplate a simplified formulation for hair conditioning shampoos which none-the-less provide improved treatment for severely dry, brittle hair. These inventions relate to or employ some steps and elements apparatus well known in the cosmetology arts and therefore, not the subject of detailed discussion h Lanodant DM erein.

Others have used gelatin in the form of hydrolyzed animal protein called collagen only as a strengthening agent in hair conditioners, and have added other ingredients for detangling and moisturizing, etc. Such formulations, typical of presently known conditioners and conditioning shampoos, are effective for detangling, but less effective for rejuvenating dry, brittle hair. Despite prior art teachings of the use of hydrolyzed animal protein, the present inventions utilize a food grade gelatin to provide effective rejuvenation of dry, brittle hair. An unexpected and surprising benefit is that this gelatin also serves as a detangling agent.

The present inventions comprise an additive amount of between 0.5% and 1.5% by weight of food grade gelatin, having specific physical properties of between 200 and 250 grams bloom; approximately 32 mps viscosity and a pH value between 4.0 and 7.0 and preferably between 4.5 and 5.8, in combination with any available basic shampoo, such as “Suave Daily Clarifying Shampoo”.

### DETAILED DESCRIPTION OF THE INVENTIONS

The present inventions are described by the following descriptions of components and methods by which the inventions can be made and used. The embodiments shown and described herein are exemplary. Many details are well known in the art, and as such are neither shown nor described.

The hair conditioner of the present inventions is made as follows:

First, one ounce food grade gelatin, having a 200-250 bloom strength and a pH value of between 4.0 and 7.0 and preferably, between 4.5 and 5.8, is stirred into 16 ounces of cold water. When the mixture becomes thick and grainy, it is heated on a stove top, or by microwave, until it completely dissolves (or liquefies). This liquefied gelatin solution is added to 88 oz. of a commercially available, plain shampoo, containing anionic and nonionic surfactants, such as “Suave Daily Clarifying Shampoo”, and blended thoroughly. Optionally, to improve shelf life, about one-half cup of a preservative, such as Lanodant DM, may be added. Also, ⅛ tsp. of red food coloring may be added to give the product a richer appearance. Another option is the addition of approximately 1 tsp. of a sheen enhancing agent, such as liquid panthenol.

The resulting product, when used as a shampoo or conditioner, with or without the above optional additives, and with no other additives, is effective for both detangling and rejuvenating dry, brittle hair. After treatment with this product, the user's hair will first seem to have an unaccustomed stiffness, but upon rinsing and drying, will have noticeably improved body and luster.

The embodiments shown and described above are exemplary. It is not claimed that all of the details, parts, elements, or steps described and shown were invented herein. Even though many characteristics and advantages of the present inventions have been described in the accompanying text, the description is illustrative only. Changes may be made in the detail, especially in matters of supplementary additives or agents, within the scope and principles of the inventions. The restrictive description of the specific examples above do not point out what an infringement of this patent would be, but are to provide at least one explanation of how to use and make the inventions. The limits of the inventions and the bounds of the patent protection are measured by and defined in the following claims.

1. A hair conditioning shampoo comprising:
a commercially available shampoo having anionic and nonionic surfactants in aqueous solution with a thickening agent; and
an amount of food grade gelatin equivalent to between 0.5% and 1.5% by weight of the aqueous solution and thickening agent, the gelatin having physical properties of approximately 200 grams bloom; approximately 32 mps viscosity and a pH value of between 4.0 and 7.0, so as to provide both detangling and rejuvenation of dry, brittle hair.

2. A hair conditioning shampoo according to claim 1 and further comprising up to 5% of a preservative agent such as Lanodant DM.

3. A hair conditioning shampoo according to claim 1 and further comprising a minor amount of a sheen enhancing agent such as Panthenol.

4. A hair conditioning shampoo according to claim 1 wherein the pH value of the gelatin is between 4.5 and 5.8.

5. The method for making a hair conditioning shampoo comprising the steps of:
stirring approximately one ounce food grade gelatin, having a 200-250 bloom strength and a pH value of between 4.0 and 7.0 into approximately 16 ounces of water and allowing the solution to rest until it becomes thick;
heating the thickened solution until it liquefies; and
blending the liquefied gelatin solution into approximately 88 oz. of a commercially available, plain shampoo, such as “Suave Daily Clarifying Shampoo”.
